(19) United States
(12) Patent Application Publication
(10) Pub. No.: US 2010/0041770 A1
(43) Pub. Date: Feb. 18, 2010

(54) POLYMER-CERAMIC COMPOSITE AND METHOD

(75) Inventors: Xinyin Liu, Exton, PA (US); Mark Thomas Fulmer, Gelnmoore, PA (US); Elliott Gruskin, Malvern, PA (US); Milvia Lepre, Allschwil (CH)

Correspondence Address: SCHWEGMAN, LUNDBERG & WOESSNER / SYNTHES, P.O. BOX 2938, MINNEAPOLIS, MN, 55402, US

(73) Assignee: Synthes USA, LLC, West Chester, PA (US)

(21) Appl. No.: 12/447,959

(22) PCT Filed: Oct. 31, 2007
(86) PCT No.: PCT/US07/23014

§ 371 Date: Oct. 7, 2009

Related U.S. Application Data
(60) Provisional application No. 60/855,904, filed on Oct. 31, 2006.

(51) Int. Cl. A61F 2/28

(52) U.S. Cl. 514/772.3; 523/116

(57) ABSTRACT

Methods and devices are shown for a composite material that is easily applied to a surface such as a bone defect in need of filling or reinforcement, etc. The composite material provides good mechanical properties such as compressive strength upon curing in the presence of water. Selected materials and methods as described are further bioabsorbable with absorption rates that are controllable to provide desired morphology over time. In selected embodiments a pharmaceutical agent further provides benefits such as bone growth, infection resistance, pain management, etc.

### RELATED APPLICATION

This patent application claims the priority benefit of U.S. Provisional Patent Application Ser. No. 60/855,904 filed Oct. 31, 2006 and entitled “IN SITU SETTING POLYMER/CERAMIC COMPOSITE BONE CEMENTS FOR CONTROLLED RELEASE OF SIMVASTATIN”, which application is incorporated herein by reference.

### BACKGROUND

The present invention relates to composite materials of ceramic and polymer. In one example the invention relates to bone replacement or void filler. In some circumstances, bones need repair, such as filling voids. In some circumstances, bones or portions of bones are replaced with artificial materials. It is desirable to use a material that is easy to put in place, and a material with desirable mechanical properties such as high strength and toughness. In some circumstances, it is also desirable for the replacement materials to be absorbed into the body, and to facilitate new bone growth in place of the absorbed material.

### BRIEF DESCRIPTION OF THE DRAWINGS

FIG. 1 is an example of a method of forming a composite material according to an embodiment of the invention.

FIG. 2 is an example of a composite material in place according to an embodiment of the invention.

FIG. 3 is an example of a delivery system and method according to an embodiment of the invention.

FIG. 4 is test data from an example embodiment of a cured composite material according to an embodiment of the invention.

FIG. 5 is test data from an example embodiment of drug release over time according to an embodiment of the invention.

FIG. 6 is test data from an example embodiment of composite material degradation over time according to an embodiment of the invention.

FIG. 7 is test data from another example embodiment of drug release over time according to an embodiment of the invention.

FIG. 8 is test data from another example embodiment of composite material degradation over time according to an embodiment of the invention.

FIG. 9 is test data from another example embodiment of drug release over time according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and minor deviations may be made without departing from the scope of the present invention.

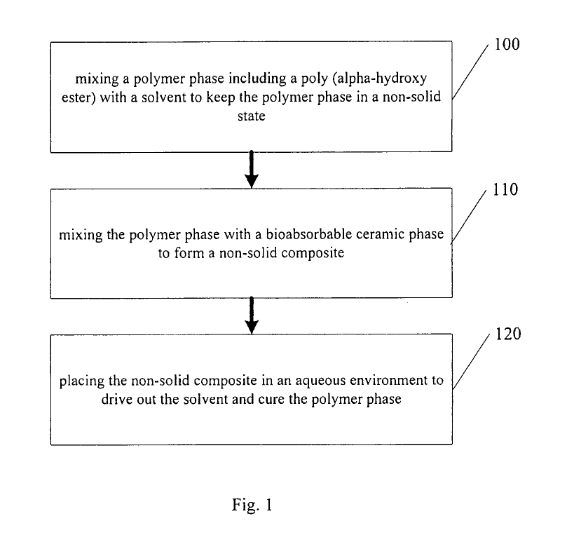

FIG. 1 shows an example method of forming a composite material. In operation 100, a polymer phase of the composite is prepared by mixing a polymer with a solvent. The example illustrated in operation 100 mixes a poly(alpha-hydroxy ester) with a solvent to keep the polymer in a non-solid state. In the present disclosure, non-solid includes a liquid, a viscous fluid, a gel, etc. In one example, having the polymer phase in a non-solid state facilitates a number of application methods for the composite material, including spreading, ejecting from a tube or syringe, etc.

A poly(alpha-hydroxy ester) is different from other polymers in that a poly(alpha-hydroxy ester) provides a polymer that can be hydrolyzed inside a patient with the hydrolyzed components being absorbed into the body. Poly(alpha-hydroxy esters) are also well researched in medical device technologies. As a result, the properties of poly(alpha-hydroxy esters) are better known than properties of other polymers. The use of poly(alpha-hydroxy esters) in patients is approved by many governing bodies such as the United States Food and Drug Administration.

Examples of acceptable poly(alpha-hydroxy esters) include but are not limited to polylactide, polyglycolide, and polycaprolactone (PCL). In one example, the polymer phase includes a copolymer where one or more portions are poly(alpha-hydroxy esters). One example includes poly(lactide-co-glycolide) and another example includes poly(lactide-co-caprolactone). Other copolymers where one or more portions are poly(alpha-hydroxy esters) include polyethylene glycol (PEG) as a component along with one or more poly(alpha-hydroxy esters) such as those listed above. Selection of an appropriate polymer phase includes identification of desired properties such as mechanical strength, adhesion to the ceramic phase, biocompatibility, bioabsorption rate, solubility in a particular solvent, etc.

As discussed above, a solvent is used with the poly(alpha-hydroxy esters) to keep the polymer phase in a non-solid state. A number of solvents are available within the scope of the invention. Example solvents are polar aprotic solvents that include, but are not limited to, n-methyl-2-pyrrolidone (NMP), 2-pyrrolidone and dimethyl sulfoxide (DMSO). Other acceptable solvents exhibit properties such as acceptable solubility of the polymer in the solvent, non-toxicity to a patient, and solubility of the solvent in water. Organic solvents such as the example solvents listed above also provide good solubility for pharmaceutical agents, such as statins that may be added to the composite material in selected embodiments described in more detail below.

In operation 110, the polymer phase and solvent are mixed with a bioabsorbable ceramic phase to form a non-solid composite such as a mixture, suspension, slurry, etc. Examples of non-solid composites include both flowable materials and moldable materials. As stated above, features of a non-solid state includes easy application and workability of the non-solid composite. In one application, a non-solid composite is pushed out of a syringe or otherwise extruded from a reservoir. Sculpting a desired shape of a composite is also possible depending on the viscosity and/or consistency of the non-solid composite.

Materials in the bioabsorbable ceramic phase include, but are not limited to various phases, physical states, and chemistries of calcium phosphate and/or calcium sulfate. In one example, a calcium phosphate cement composition is used as the bioabsorbable ceramic material.

Some specific examples of calcium phosphates and calcium sulfates include, but are not limited to: crystalline calcium phosphates or calcium sulfates; dicalcium phosphate anhydrous-CaHPO₄; dicalcium phosphate dihydrate-CaHPO₄.2H₂O; α-tricalcium phosphate-Ca₃(PO₄)₂; α′-tricalcium phosphate-Ca₃(PO₄)₂; β-tricalcium phosphate-Ca₃(PO₄)₂; hydroxyapatite-Ca₅(PO₄)₃OH, or Ca₁₀(PO₄)₆(OH)₂; tetracalcium phosphate-Ca₄ (PO₄)₂O; octacalcium phosphate-Ca₈H₂(PO₄)₆.5H₂O; calcium sulfate anhydrous-CaSO₄; α-calcium sulfate hemihydrate-α-CaSO₄.½H₂O; β-calcium sulfate hemihydrate-β-CaSO₄.½H₂O; or calcium sulfate dihydrate-CaSO₄.2H₂O containing cements. Although a number of example compositions and phases are listed, other compositions and phases of calcium phosphate and/or calcium sulfate are within the scope of the invention.

In operation 120, the non-solid composite is placed in an aqueous environment. In one example method, a patient is having a bone repaired or replaced. A void or other defect, for example, can be filled with the non-solid composite. The environment inside a patient contains sufficient water to be included in an aqueous environment in the present disclosure. In such an example, the biological fluids in a patient that surrounds the non-solid composite drives out the solvent from the polymer. The polymer then precipitates or otherwise hardens within the composite material to form a solid material. As discussed above, in one embodiment, the solvent is easily absorbed into the body as it is diffused out.

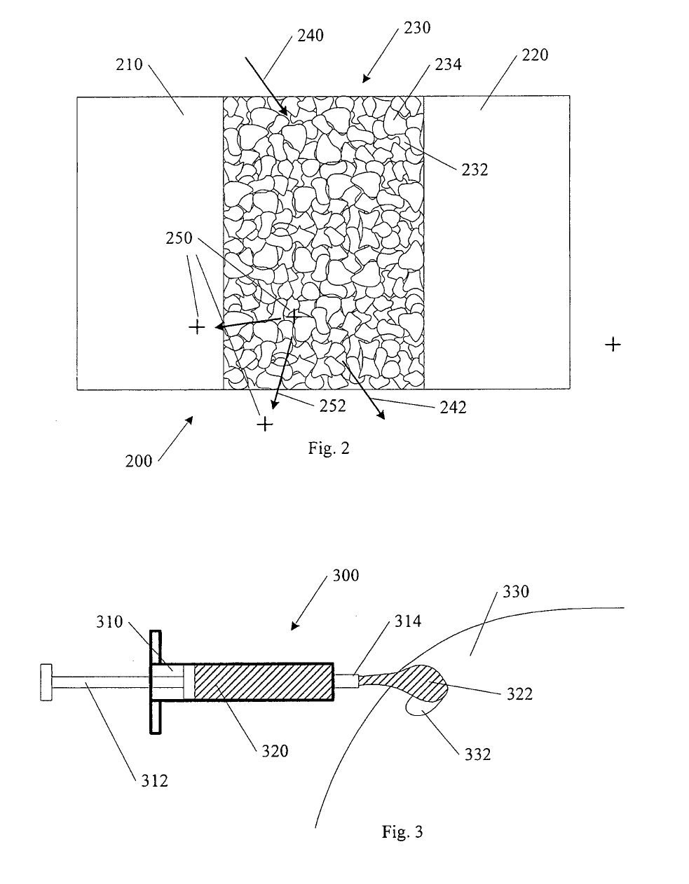

One example of a resulting solid composite structure is shown in FIG. 2. A first existing bone portion 210 and a second existing bone portion 220 are shown with a solid composite structure 230. The composite structure 230 includes a polymer phase 232 and a bioabsorbable ceramic phase 234. In the example shown, the bioabsorbable ceramic phase 234 is dispersed within the polymer phase 232 matrix.

As discussed above, in one example the composite structure 230 is applied to a desired location, such as between the first existing bone portion 210 and a second existing bone portion 220 in a non-solid state. Once in place, the composite structure 230 is cured as water diffuses into the structure as shown by arrow 240, and the solvent diffuses out of the structure as shown by arrow 242. In one example a resulting composite structure formed from poly (DL-lactide) and calcium phosphate cement in a ratio of 1:3 respectively provided a compressive strength of 3-5 MPa after curing for 24 hours at approximately 37 degrees C.

After the composite structure 230 is cured, one method includes degrading the composite structure 230 over time to be bioabsorbed into the body of the patient while the composite structure 230 is replaced by new bone growth. In one embodiment, a bioabsorption rate of the ceramic phase is compared to a bioabsorption rate of the polymer phase. In one example, the bioabsorption rate of the polymer phase is controlled by varying a molecular weight of the polymer phase. Other methods of controlling the bioabsorption rate of the polymer phase are also within the scope of the invention. In one embodiment, a bioabsorption rate of the ceramic phase is also controlled.

In one embodiment, the respective rates of bioabsorption are controlled within the composite to achieve a desired bone growth mechanism. One method includes adjusting the bioabsorption rate of the polymer phase to approximately match the bioabsorption rate of the ceramic phase. Matching rates of bioabsorption reduce the possibility of leaving behind a pocked or holed structure where one of the phases has been absorbed faster than the other. In other methods, a pocked or holed structure is desired to provide nucleation sites for new bone growth.

In one embodiment, a hydrophilic agent is included in the polymer phase of the composite to adjust the respective rates of bioabsorption as noted above. In selected embodiments, the hydrophilic agent includes a hydrophilic oligomer or polymer. Hydrophilic agents, including oligomers or polymers, etc. are absorbed more readily than other components in the composite material, leaving pores behind in the composite.

Examples of hydrophilic agents include polyvinyl alcohol (PVA), polyvinyl pyrrolidone (PVP), polyethylene glycol (PEG), and polyethylene oxide (PEO), etc. Other examples of hydrophilic agents include oligosacchrides, polysacchrides and their derivatives, such as dextran, alginate, hyaluronate, carboxymethyl cellulose, hydroxypropyl methyl cellulose or other cellulose derivatives.

As discussed above, in selected embodiments pores are desirable, and used to adjust parameters such as available nucleation sites for replacement bone growth and exposed surface area, which is related to rate of release of other included elements such as pharmaceutical agent (discussed in more detail below).

While hydrophilic polymers are described, other materials that are included in the composite material to control rate of porosity are within the scope of the invention. Using the polymer example, hydrophilic polymers can be included in the composite material by a number of possible mechanisms including, but not limited to, copolymerization, physical blending, etc.

In one embodiment, a pharmaceutical agent 250 is included within the composite structure 230. One example of a pharmaceutical agent 250 includes a bone growth promoting agent. A statin such as simvastatin is an example of a pharmaceutical agent that has been shown to promote bone growth. In one embodiment a hydrophobic pharmaceutical agent such as a statin is dissolved in an organic solvent such as n-methyl-2-pyrrolidone (NMP), 2-pyrrolidone or dimethyl sulfoxide (DMSO) as discussed above. An advantage of such a solvent/pharmaceutical agent combination includes a more reproducible drug release profile as the composite material degrades, due to more even distribution of the pharmaceutical agent within the composite material. In selected embodiments, such a property is desirable to minimize rapid release of the pharmaceutical agent and to prolong the release profile.

Other bone growth promoting agents that may be included within the composite structure 230 include, but are not limited to, proteins or peptides that are related to bone formation, healing and repair. Examples of proteins include bone morphogenic proteins (BMPs), osteogenic proteins (OP), transforming growth factors (TGF), insulin-like growth factor (IGF), platelet-derived growth factor (PDGF), vascular endothelial growth factor (VEGF).

Other pharmaceutical agents that may be included within the composite structure 230 include antibiotics, analgesics, and cancer drugs, or a combination of any agents listed above. In one embodiment, a pharmaceutical agent 250 or agents are contained within the polymer phase 232 of the composite structure 230, although the invention is not so limited. Other examples of composite structures 230 include pharmaceutical agents in the ceramic phase, or both the polymer and the ceramic phase.

In one embodiment the pharmaceutical agent 250 diffuses out of the composite structure 230 and into surrounding tissue or into adjacent bone over time as shown by arrows 252. In one example the pharmaceutical agent 250 is released as the composite structure 230 degrades. In one embodiment where the pharmaceutical agent 250 is contained within the polymer phase, a ratio of polymer phase to ceramic phase controls a rate of release of the pharmaceutical agent 250.

FIG. 3 illustrates one example of a delivery system 300 according to an embodiment of the invention. A storage chamber 310 is illustrated with a quantity of non-solid composite material 320 as described in embodiments above contained within the storage chamber 310. In the example shown, the delivery system 300 includes a syringe, although the invention is not so limited. In operation, a plunger 312 is pressed to dispense the non-solid composite material 320 from the storage chamber 310 out through a nozzle 314.

FIG. 3 illustrates using the delivery system 300 to fill a void 332 in a bone surface 330 such as a skull for example. A quantity 322 of the non-solid composite material 320 fills in the void 332 while in the non-solid state. As described above, in one embodiment, biological fluids from the patient tissue drives out the solvent within the polymer phase of the non-solid composite material 320 and cures the composite into a solid.

In one example the non-solid composite material 320 is stored within the storage chamber 310 in the non-solid state until needed. Upon application, the composite material then cures. In other examples, the non-solid composite material 320 is prepared just before a procedure from components such as polymer, solvent, and ceramic. The non-solid composite material 320 is then applied and cured in place.

Using composite materials and methods as described, a composite material is easily applied to a portion of bone in need of filling or reinforcement, etc. The composite material provides good mechanical properties such as compressive strength upon curing. Selected materials and methods as described are further bioabsorbable with absorption rates that are controllable to provide a desired effect. In selected embodiments a pharmaceutical agent further provides benefits such as bone growth and formation, infection resistance, pain management, etc.

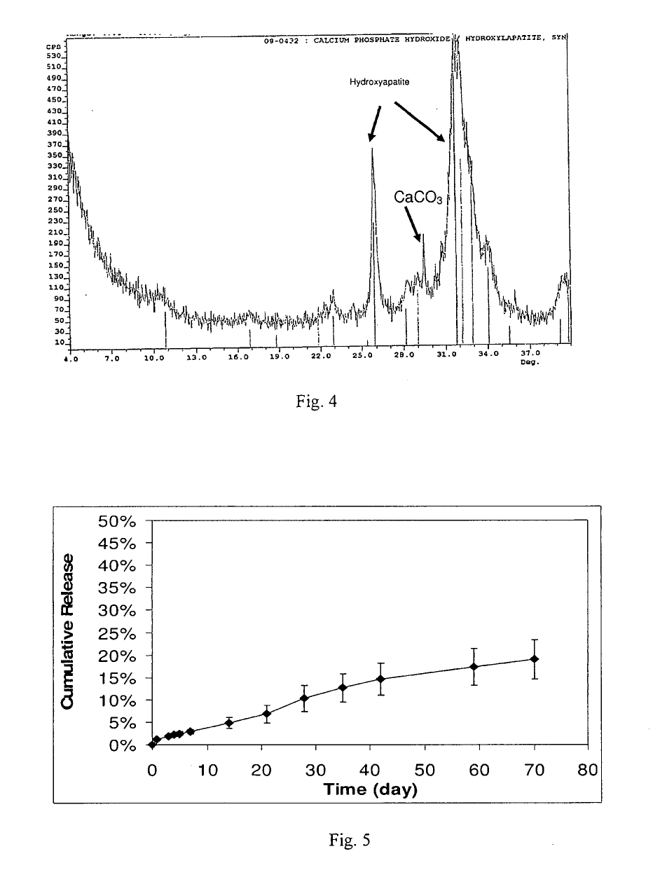

FIGS. 4-9 show selected test data from example embodiments. The materials, such as polymers, ceramic phases, and solvents shown are illustrated as examples only. Likewise, the specific preparation and test methods are shown as examples only. The scope of the invention includes any other materials or combination and methods as determined with reference to the appended claims, along with the full scope of equivalents to which such claims are entitled.

FIG. 4 illustrates X-ray diffraction spectra of the PLGA/calcium phosphate cement in phosphate buffered saline (PBS) (pH 7.4) at 37° C. for 1 week. The test sample was prepared and evaluated as follows. PLGA (50/50, i.v.=0.48 dl/g) was dissolved in NMP at weight ratio of 1:2. 3 g calcium phosphate cement powder was then mixed with 6 g of PLGA-NMP to form a paste-like mixture, which was injected through a 3 mL oral syringe with an opening of 3 mm into phosphate buffered saline (pH 7.4) at 37° C. for 1 week. The mixture started to harden in contact with PBS. By the end of 1 week, calcium phosphate cement cured into hydroxyapatite with trace calcium carbonate (FIG. 4), which resembles the bone mineral phase.

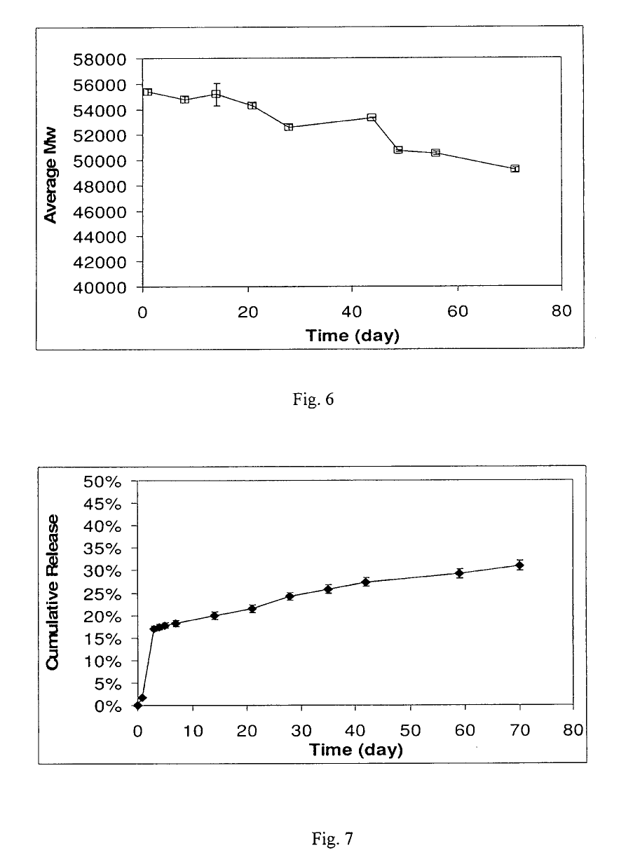

FIG. 5 illustrates cumulative release of simvastatin from 1:1 (wt) PDLLA (i.v.=0.49 dl/g)/calcium phosphate cement in PBS (pH 7.4) at 37° C. for 10 weeks (n=3). FIG. 6 illustrates degradation of the same test sample. The example was prepared and evaluated as follows. PDLLA (i.v.=0.49 dl/g) was dissolved in NMP at weight ratio of 1:2. 0.3 g of simvastatin was first mixed with 5 g of PDLLA-NMP, and then 5 g calcium phosphate cement powder was added to form a paste-like mixture, which was injected through a 3 mL oral syringe with opening of 3 mm. Release studies were performed in phosphate buffered saline (pH 7.4) at 37° C. for 10 weeks (FIG. 5). The concentration of simvastatin was measured with reverse phase high performance liquid chromatography (HPLC) equipped with a photodiode array (PDA) detector. The degradation of PDLLA (i.v.=0.49 dl/g) was measured using gel permeation chromatography (GPC) polystyrene as narrow standards (FIG. 6).

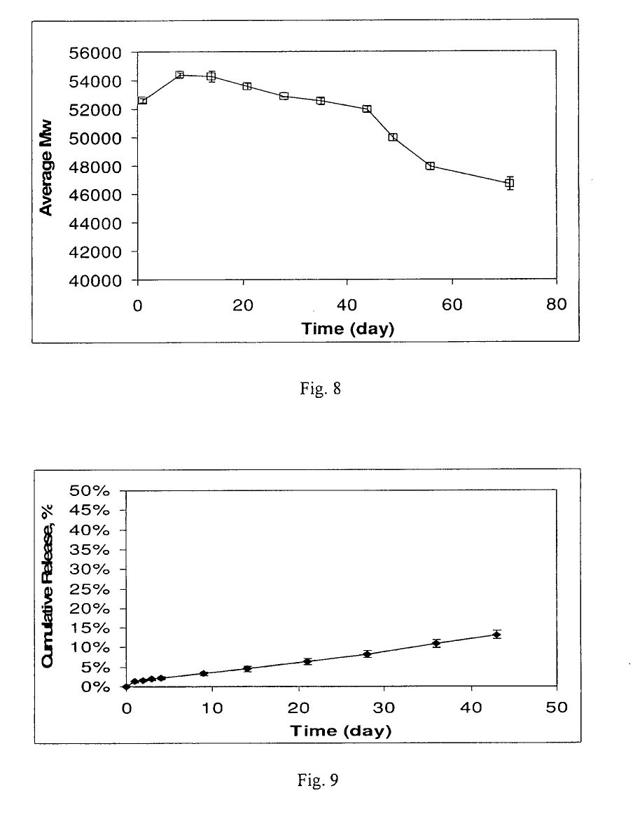

FIG. 7 illustrates cumulative release of simvastatin from 2:1 (wt) PDLLA (i.v.=0.49 dl/g)/calcium phosphate cement in PBS (pH 7.4) at 37° C. for 10 weeks (n=3). FIG. 8 illustrates degradation of the same test sample. The example was prepared and evaluated as follows. PDLLA (i.v.=0.49 dl/g) was dissolved in NMP at a weight ratio of 1:2. 0.27 g of simvastatin was first mixed with 6 g of PDLLA-NMP, and then 3 g calcium phosphate cement powder was added to form a paste-like mixture, which was injected through a 3 mL oral syringe with opening of 3 mm. Release studies were performed in phosphate buffered saline (pH 7.4) at 37° C. for 10 weeks (FIG. 7). The concentration of simvastatin was measured with reverse phase high performance liquid chromatography (HPLC) equipped with a photodiode array (PDA) detector. The degradation of PDLLA (i.v.=0.49 dl/g) was measured using gel permeation chromatography (GPC) polystyrene as narrow standards (FIG. 8).

FIG. 9 illustrates results of cumulative release of simvastatin from 4:1 (wt) PDLLA (i.v.=1.87 dl/g)/calcium phosphate cement in PBS (pH 7.4) at 37° C. for 6 weeks (n=3) according to one example embodiment. The example was prepared and evaluated as follows. PDLLA (i.v.=1.87 dl/g) was dissolved in NMP at a weight ratio of 1:4. 0.23 g of simvastatin was first mixed with 6 g of PDLLA-NMP, and then 1.5 g calcium phosphate cement powder was added to form a paste-like mixture, which was injected through a 3 mL oral syringe with an opening of 3 mm. Release studies were performed in phosphate buffered saline (pH 7.4) at 37° C. for 6 weeks (FIG. 9). The concentration of simvastatin was measured with reverse phase high performance liquid chromatography (HPLC) equipped with a photodiode array (PDA) detector.

While a number of example embodiments and advantages of the invention are described, the above examples are not exhaustive, and are for illustration only. Although specific embodiments have been illustrated and described herein, it will be appreciated by those of ordinary skill in the art that any arrangement or method which is calculated to achieve the same purpose may be substituted for the specific embodiment shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and methods are used. The scope of the invention should be determined with reference to the appended claims, along with the full scope of equivalents to which such claims are entitled.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A composite material, comprising:
a polymer phase including a poly(alpha-hydroxy ester) mixed with a solvent to keep the polymer phase in a non-solid state; and
a bioabsorbable ceramic phase mixed with the polymer phase;
wherein when in the presence of water, the solvent is diffused out of the polymer phase to cause solidification of the polymer phase and curing of the composite material.

2. The composite material of claim 1, wherein the solvent is chosen from a group consisting of n-methyl-2-pyrrolidone, 2-pyrrolidone, and dimethyl sulfoxide.

3. The composite material of claim 1, wherein the poly(alpha-hydroxy ester) includes one ore more of polylactide, polycaprolactone, a copolymer including poly(lactide-co-glycolide), a copolymer including polycaprolactone and polylactide, a copolymer including a polyethylene glycol and one or more poly(alpha-hydroxy esters) chosen from a group consisting of polycaprolactone, polylactide, and polyglycolide.

4. The composite material of claim 1, wherein the polymer phase includes a copolymer.

5. The composite material of claim 1, wherein the polymer phase includes a physical blend of a poly(alpha-hydroxy ester) and one or more hydrophilic agents.

6. The composite material of claim 1, wherein the bioabsorbable ceramic includes one or more of calcium phosphate, calcium sulfate, and a mixture of calcium phosphate and calcium sulfate.

7. The composite material of claim 1, wherein the composite material is contained in a non-solid state in a storage chamber within a delivery device.

8. The composite material of claim 7, wherein the delivery device includes a syringe to keep the composite material in the non-solid state prior to delivery.

9. The composite material of claim 7, wherein the composite material is flowable prior to curing or moldable prior to curing.

10. The composite material of claim 1, further including a pharmaceutical agent within the composite material to release over time from the composite material.

11. The composite material of claim 10, wherein the pharmaceutical agent is within the polymer phase.

12. The composite material of claim, wherein the pharmaceutical agent includes an agent promoting bone growth, remodeling and healing.

13. The composite material of claim 10, wherein the pharmaceutical agent chosen from group consisting of antibiotics, analgesics, statins, cancer drugs.

14.-26. (canceled)

27. A method, comprising:
mixing a polymer phase including a poly(alpha-hydroxy ester) with a solvent to keep the polymer matrix in a non-solid state;
mixing the polymer phase with a bioabsorbable ceramic phase to form a non-solid composite;
placing the non-solid composite in an aqueous environment to drive out the solvent and cure the polymer phase.

28. The method of claim 27, wherein placing the non-solid composite in an aqueous environment includes dispensing the non-solid composite from a delivery device into an aqueous environment.

29. The method of claim 27, wherein the mixing of the polymer phase with the bioabsorbable ceramic phase is performed just prior to placing the non-solid composite in the aqueous environment.

30. The method of claim 27, wherein mixing the polymer phase including the poly(alpha-hydroxy ester) with the solvent includes mixing a polymer phase including a poly(alpha-hydroxy ester) with n-methyl-2-pyrrolidone.

31. The method of claim 27, wherein mixing the polymer phase including the poly(alpha-hydroxy ester) with the solvent includes mixing a polymer phase including a poly(alpha-hydroxy ester) with dimethyl sulfoxide.

32.-35. (canceled)

36. The method of claim 27, wherein mixing the polymer phase includes mixing a physical blend of poly(alpha-hydroxy esters) with polyethylene glycol.

37. The method of claim 27, wherein mixing the polymer phase includes mixing a physical blend of poly(alpha-hydroxy esters) with polyethyelene oxide.

38.-39. (canceled)

40. The method of claim 27, wherein mixing the polymer phase with the bioabsorbable ceramic phase includes mixing the polymer phase with calcium phosphate.
